(19) United States
(12) Patent Application Publication
(10) Pub. No.: US 2011/0015527 A1
(43) Pub. Date: Jan. 20, 2011

(54) FLAT DOPPLER PROBE AND METHOD OF THE SAME

(75) Inventors: Ray Heasty, McFarland, WI (US); Tony Poole, Tetbury (GB); Christina Zeisler, Cambridge, WI (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: Cardinal Health - Neurocare, Madison, WI (US)

(21) Appl. No.: 12/503,352

(22) Filed: Jul. 15, 2009

(51) Int. Cl. A61B 8/13

(52) U.S. Cl. 600/459

(57) ABSTRACT

A flat ultrasound probe includes a housing having sidewalls, each having a height, a bottom surface for contacting an external surface of a patient during operation of the probe, the bottom surface having a width larger than the height of the sidewalls and a flat portion, and a recession in the bottom surface for containing a transmission material on an outer surface of the housing for aiding in transmission of ultrasound signals, the recession being rounded on all sides where the recession contacts the flat portion of the bottom surface.

### FIELD OF THE INVENTION

The present invention relates to ultrasound probes, that are generally flat probes. More specifically, the present invention relates specifically to Doppler probes.

### BACKGROUND OF THE INVENTION

Ultrasound scanning, also commonly referred to as sonography, is oftentimes used to view and/or examine tissues and organs inside the body. It employs high-frequency sound waves, which cannot be heard by humans, to produce images of structures inside the body. Sonography allows for the production of images of organs that are soft or filled with fluid, but it is less effective for examining air-filled organs or bones.

One of the most common uses of sonography is to evaluate the progress of the fetus during pregnancy. Another common use of sonography is to view and to determine whether a lump or mass is a cyst. Moreover, sonography is utilized to look at the size and shape of abdominal and pelvic organs, to detect gallstones, and to detect blood clots in the legs. It can also be utilized as a guide when a needle is being inserted into the body to take a sample of tissue for a biopsy or to take a fluid sample, for example, as is done in amniocentesis, a test to detect abnormalities in the fetus.

Typically, ultrasound probes are used in combination with a small amount of transmission material, e.g. a gel, will be applied on the skin over the area to be scanned to help the sound waves transmit into a patient's body. The doctor or ultrasound technician typically slides or translates the ultrasound instrument back and forth through this gel. During the monograph, the ultrasound instrument, also called a transducer, transmits ultrasound waves into the patient's body there the waves reflect, or echo, when they contact organs, bone, or similar tissue. The reflected sound waves are then received by the transducer, processed by a computer, and transmitted to a lighted screen to produce an image.

Another common use of sonography is Doppler ultrasound which is an important technique for non-invasively measuring the velocity of moving structures, particularly blood within the body. A flat Doppler probe is typically employed and/or preferred when a signal is required for extended periods of time and the user is unable or unwilling to hold the probe for the duration of the test. The Doppler signals may be used, e.g., to determine blood flow, direction of blood flow, and/or generate audio signals based on the blood flow. For example, it is typically more difficult to obtain Doppler signals on patients with vascular disease because of their decreased blood flow. One type of Doppler probe is a continuous-wave probe which may have two crystals: one for transmitting Doppler signals and one for receiving reflected signals.

A drawback of conventional flat Doppler probes is that the Doppler crystal is not positioned at an optimum angle for ease of use (sensitivity for signal location). In addition, the flat surface does not leave room for enough gel to be applied to aid in receiving the Doppler signals.

Accordingly, there is a need and desire to provide a flat Doppler probe with a Doppler crystal positioned at an optimum angle for ease of use and allowing enough gel to be applied to aid in receiving the Doppler signals.

### SUMMARY OF THE INVENTION

Embodiments of the present invention advantageously provide a flat ultrasound probe with an ultrasound crystal at an optimum angle for ease of use and allowing enough gel to be applied to aid in receiving the ultrasound signals.

An embodiment of the invention includes a flat ultrasound probe which includes a housing having sidewalls, each having a height, a bottom surface for contacting an external surface of a patient during operation of the probe, the bottom surface having a width larger than the height of the sidewalls and a flat portion, and a recession in the bottom surface for containing a transmission material on an outer surface of the housing for aiding in transmission of ultrasound signals, the recession being rounded on all sides where the recession contacts the flat portion of the bottom surface.

Another embodiment of the invention includes a method of providing an ultrasound Doppler spectrum using a flat ultrasound probe, the method including producing an original ultrasound signal with a flat ultrasound probe, the probe including a housing, including sidewalls, each having a height, a bottom surface for contacting an external surface of a patient during operation of the probe, the bottom surface having a width larger than the height of the sidewalls and a flat portion, and a recession in the bottom surface for containing a transmission material on an outer surface of the housing, the recession being rounded on all sides where the recession contacts the flat portion of the bottom surface. The method further includes receiving a reflected ultrasound signal, and generating a Doppler spectrum on a display based on the received reflected ultrasound signal.

Another embodiment of the invention includes a flat ultrasound probe, including means for housing, including sidewalls, each having a height, bottom means for contacting an external surface of a patient during operation of the probe, the bottom means having a width larger than the height of the sidewalls and a flat portion, and recession means in the bottom means on an outer surface of the housing means for containing a means for aiding transmission for aiding in transmission of ultrasound signals, the recession means being rounded on all sides where the recession means contacts the flat portion of the bottom means.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The above-mentioned and other features and advantages of this disclosure, and the manner of attaining them, will become more apparent and the disclosure itself will be better understood by reference to the following description of various embodiments of the disclosure taken in conjunction with the accompanying figures, wherein:

FIG. 1 is a bottom schematic view of a flat probe in accordance with an embodiment of the present invention.

FIG. 2A is a side schematic view of a flat probe in accordance with an embodiment of the present invention.

FIG. 2B is a top schematic view of a flat probe in accordance with an embodiment of the present invention.

FIG. 3 is a rear schematic view of the FIG. 2A probe taken along the line A-A′.

FIG. 4A is a side schematic view of a flat probe in accordance with an embodiment of the present invention.

FIG. 4B is an expanded view of a section of the FIG. 4A probe.

FIG. 5 is a top view of a portion of a flat probe in accordance with an embodiment of the present invention.

### DETAILED DESCRIPTION OF THE INVENTION

In the following detailed description, reference is made to the accompanying drawings, which form a part hereof and show by way of illustration specific embodiments in which the invention may be practiced. These embodiments are described in sufficient detail to enable those skilled in the art to practice them, and it is to be understood that other embodiments may be utilized, and that structural, logical, processing, and electrical changes may be made. It should be appreciated that any list of materials or arrangements of elements is for example purposes only and is by no means intended to be exhaustive. The progression of processing steps described is an example; however, the sequence of steps is not limited to that set forth herein and may be changed as is known in the art, with the exception of steps necessarily occurring in a certain order.

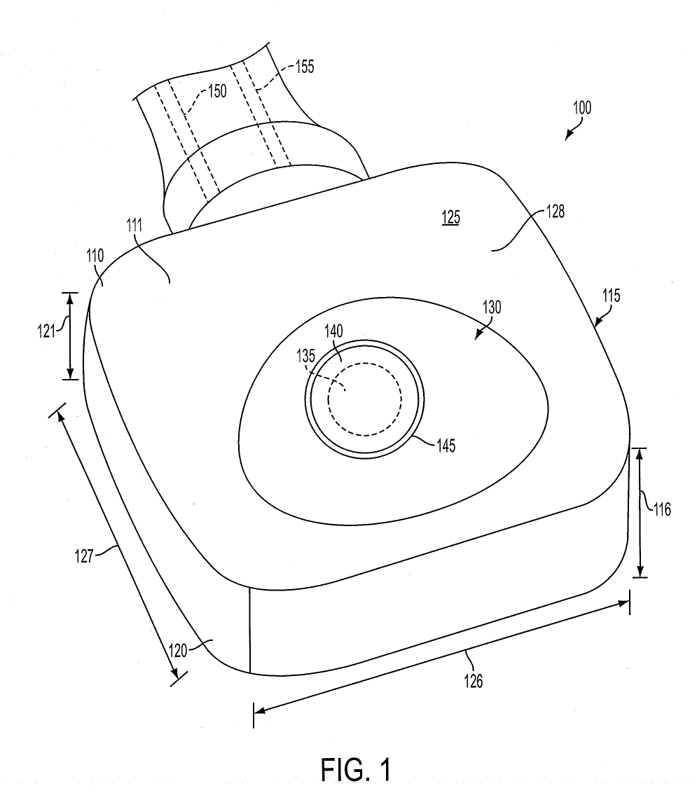

The invention will now be described with reference to the drawing figures in which like reference numerals refer to like parts throughout. As depicted in FIG. 1, a flat ultrasound Doppler probe 100 is depicted comprising a housing 110 having sidewalls 115, 120 each having a respective height 116, 121. The housing further includes a bottom surface 125 for contacting an external surface of a patient or the like (not shown) during operation of the probe 100. The bottom surface 125, e.g., the working surface, preferably has a width 126 larger than the height of the sidewalls 116, 121 and has a flat portion 128. The sidewall heights 116, 121 may be the same height or varying height. As can be seen in FIG. 1, a recession 130 is located in the bottom surface 125. The recession 130 functions to retain a transmission material, e.g. a gel, on an outer surface 111 of the housing 110. The gel functions to aid in transmission of ultrasound signals during operation of the probe 100. The recession 130 is rounded on all sides where the recession 130 contacts the flat portion 128 of the bottom surface 125. The shape of the recession 130 is optimized for cleaning the gel out of the recession 130, preferably being large enough for an operator to insert a finger. The probe 100 also has at least one crystal 135 located inside the housing 110 behind the recession 130, preferably at an angle for providing an ultrasound signal. The angle may be manipulated to optimize the receipt of a more sensitive Doppler reading.

The recession 130 may have a geometry based on the angle of the crystal 135 to optimize a path for the Doppler signal. For example, the recession 130 may have an inner surface 140 having a conical geometry. The crystal 135 may be positioned behind the thinnest part of the probe 100, such as behind the inner surface 140, so that a minimal amount of the material from which the probe is constructed is between the crystal and the transmission material (e.g., gel). This may further aid in transmission and reception of Doppler signals. A fillet 145 may be placed around the perimeter of the inner surface 140 to aid in manufacturing the probe 100 via injection molding while leaving enough room for the Doppler crystal 135 or crystals. The fillet 145 may be oriented, for example, at a 30 degree angle with respect to the bottom surface 125. In one embodiment, the bottom surface 125 may have a length 127 within 15% of the width 126, such that the bottom surface 125 has a generally square cross section. However, any other geometry may be used, so long as the crystals, e.g., crystal 135, and internal circuitry (depicted in FIG. 5 as reference number 515) are not adversely affected. The term “flat” refers to the bottom surface 125 being generally flat, except for the recessed portion 130 for holding the gel. The probe 100 may have both a control signal transmitting line 150 and a control signal receiving line 155 for sending control signals to and from the probe 100. The probe 100 may operate in the 8 MHz ultrasound range, although it should be appreciated that any other frequency or frequency band may be used, as appropriate. The crystal generates the original ultrasound signal and the probe receives a reflected signal which is used to generate an ultrasound Doppler spectrum on a display.

The gel cavity, e.g., recession 130, on the flat Doppler probe 100 positions the Doppler crystal 135 at an optimum angle for ease of use (e.g., sensitivity for the signal location), as previously described. The geometry of the recession 130 enables a user to quickly and thoroughly clean the recession 130 after use. This accessible recession 130 is an improvement over existing flat probes for sensitivity and ease of cleaning.

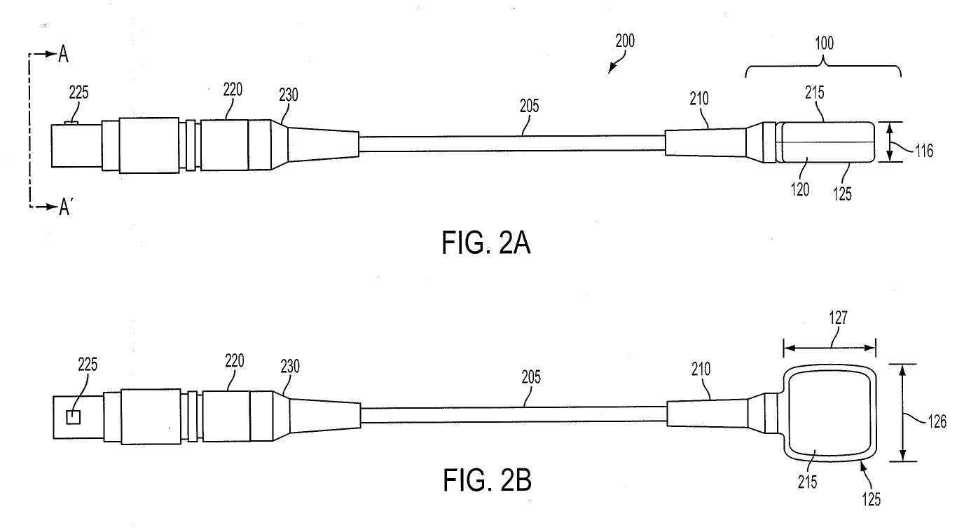

Turning now to FIG. 2A, it shows a probe assembly 200 with the probe 100 attached to a cable 205 at an optional first bend relief connection 205. The probe 100 is illustrated in FIG. 2A with a top surface 215 and the bottom surface 125. The height 116 of one of the sidewalls 115 is also shown. The cable 205 may be of any length and/or type appropriate for reaching between the patient and a control system (not shown). The cable 205 attaches to the control system by an optional connector 220 which may have a connector release 225. It should be appreciated that the connector release 225 may be on any side of the connector 220, as appropriate for ease of use. The cable 205 may be attached to the connector by an optional second bend relief connection 230.

FIG. 2B depicts the probe assembly 200 from a top view showing the top surface 215 of the probe 100. The length 127 and width 126 of the bottom surface 125 is also illustrated. The top surface 215 may have the same length 127 and width 126 as the bottom surface 125, or may be different, depending upon application and/or preference.

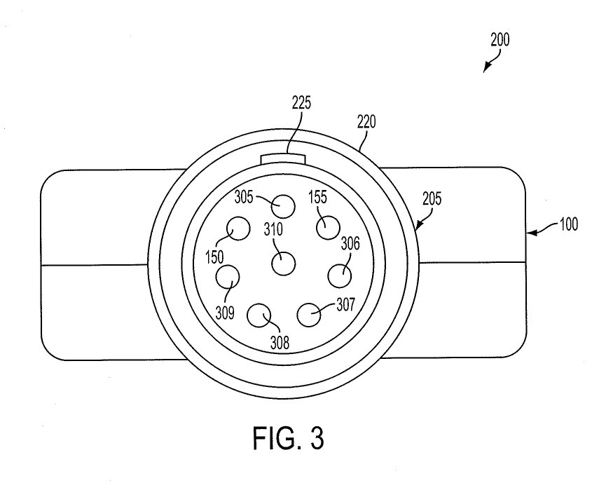

Turning to FIG. 3, it provides a rear schematic view of the FIG. 2A probe taken along the line A-A′. In addition to the control signal transmitting line 150 and the control signal receiving line 155, additional signal lines 305-310 may be arranged through the cable 205. It should be appreciated that more or fewer signal lines may be used, where appropriate.

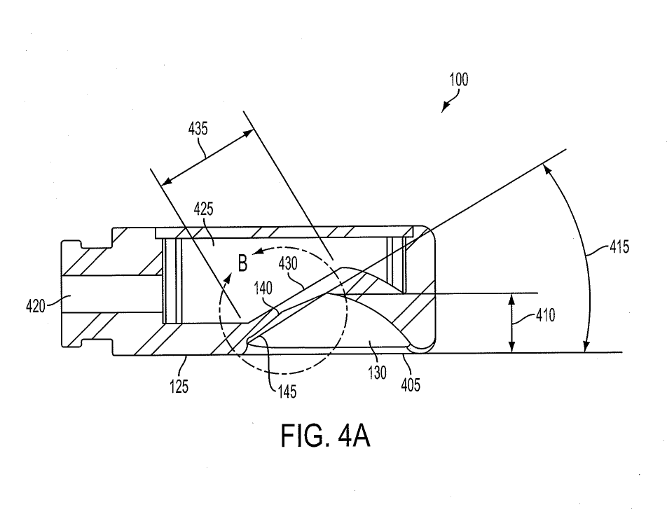

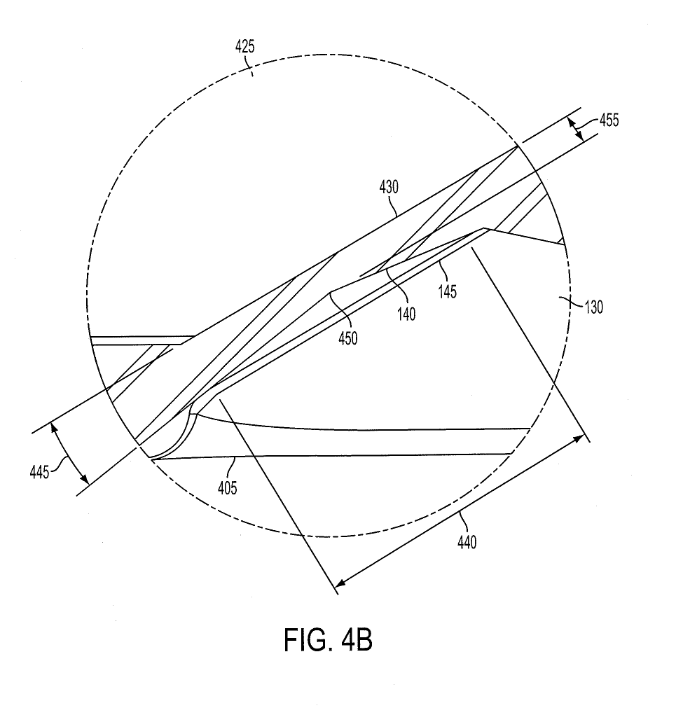

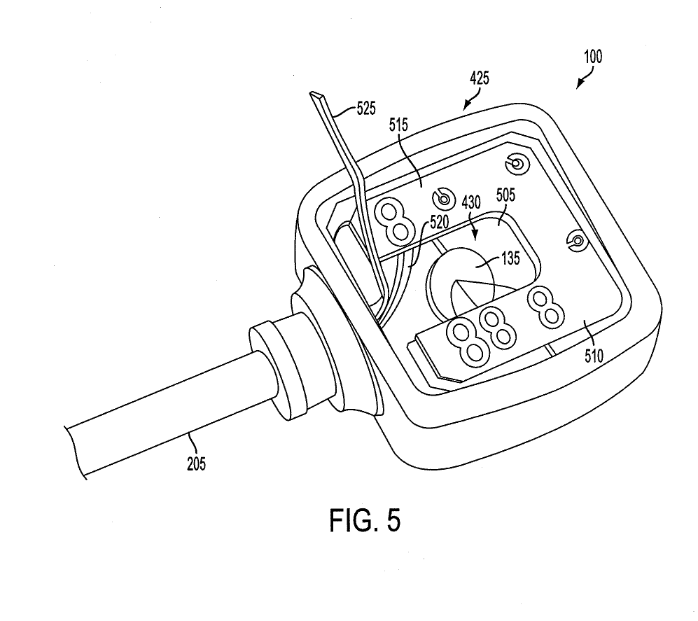

FIG. 4A depicts a cross section of the probe 100. The recession 130 contacts the bottom surface 125 at a recession perimeter 405. The deepest point of the recession 130 may be at a location where the fillet 145 is furthest from the perimeter 405, having a depth 410. The fillet may be tilted at an angle 415, which may be, for example, 30 degrees from the bottom surface 125. The control signal transmitting line 150 and the control signal receiving line 155 may be arranged through an opening 420 to an inner area 425 which holds the crystal 135 and internal circuitry 515 (FIG. 5), both of which will be shown in more detail in FIG. 5. The inner area 425 has a flat surface 430 against which the crystal 135 is placed upon assembly. The flat surface 430 has a width 435 and is substantially parallel to the fillet 145.

As illustrated in FIG. 4B, it shows the circular area defined by line B in greater detail. The width 440 of the fillet 145 may be defined where the fillet 145 meets the inner surface 140 of the recession 130. The inner surface 140 may be pitched at an angle 445 with respect to the flat surface 430 of the inner area 425. The deepest point 450 of the inner surface 140 may have a distance 455 from the flat surface 430 of the inner area 425 which may be optimized for strength of the probe 100 while allowing enough space for the ultrasound transmission material, e.g., gel.

Turning to FIG. 5, it depicts the inner area 425 of the probe 100 from a top view without the top surface 215 attached. As can be seen, the crystal 135 is placed over the flat surface 430. A shield 505 is placed around the crystal 135 for ensuring that signals inside the probe 100 do not interfere with the ultrasound signal generated by the crystal 135. Internal circuitry 510 is arranged over the shield 505 and around the crystal 135. The internal circuitry 510 may be held, e.g., on a printed circuit board 515. Signals may be sent to and from the internal circuitry 510 by signal line 520, which may be, for example, any of the control signal transmitting line 150, the control signal receiving line 155, and the additional signal lines 305-310. An optional grounding line 525 may be included for reducing interference and/or ensuring the probe is properly electrically grounded.

The processes and devices in the above description and drawings illustrate examples of only some of the methods and devices that could be used and produced to achieve the objects, features, and advantages of embodiments described herein. Thus, they are not to be seen as limited by the foregoing description of the embodiments, but only limited by the appended claims. Any claim or feature may be combined with any other claim or feature within the scope of the invention.

The many features and advantages of the invention are apparent from the detailed specification, and, thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and, accordingly, all suitable modifications and equivalents may be resorted to that fall within the scope of the invention.

What is claimed is:

1. A flat ultrasound probe, comprising:
a housing, comprising:
sidewalls, each having a height;
a bottom surface for contacting an external surface of a patient during operation of the probe, the bottom surface comprising:
a width larger than the height of the sidewalls; and
a flat portion; and
a recession in the bottom surface for containing a transmission material on an outer surface of the housing for aiding in transmission of ultrasound signals, the recession being rounded on all sides where the recession contacts the flat portion of the bottom surface.

2. The probe of claim 1, further comprising:
a crystal located inside the housing at an angle for providing an ultrasound signal,
wherein the recession comprises a geometry based on the angle of the crystal.

3. The probe of claim 2, wherein the recession comprises an inner surface having a conical geometry.

4. The probe of claim 3, wherein the crystal is located at a thinnest portion of the inner surface to optimize Doppler signal passage.

5. The probe of claim 4, wherein the recession further comprises a fillet surrounding the inner surface.

6. The probe of claim 5, wherein the fillet is arranged at a 30 degree angle with respect to the bottom surface.

7. The probe of claim 1, further comprising internal circuitry for controlling the crystal.

8. The probe of claim 7, further comprising transmitting and receiving lines for sending signals to and from the internal circuitry.

9. The probe of claim 1, wherein the probe is configured to operate in an ultrasound range near 8 MHz.

10. The probe of claim 1, further comprising:
a cable for protecting the transmitting and receiving lines;
a connector for attaching the cable to a control system; and
a connector release for releasing the connector from the control system.

11. A method of providing an ultrasound Doppler spectrum using a flat ultrasound probe, the method comprising:
producing an original ultrasound signal with a flat ultrasound probe, the probe comprising:
a housing, comprising:
sidewalls, each having a height;
a bottom surface for contacting an external surface of a patient during operation of the probe, the bottom surface comprising:
a width larger than the height of the sidewalls; and
a flat portion; and
a recession in the bottom surface for containing a transmission material on an outer surface of the housing, the recession being rounded on all sides where the recession contacts the flat portion of the bottom surface;
receiving a reflected ultrasound signal; and
generating an Doppler spectrum based on the received reflected ultrasound signal.

12. The method of claim 11, wherein:
the housing further comprises a crystal inside the housing at an angle for providing the original ultrasound signal; and
the recession is provided with a geometry based on the angle of the crystal.

13. The method of claim 12, wherein the recession comprises an inner surface having a conical geometry.

14. The method of claim 13, wherein the crystal is located at a thinnest portion of the inner surface to optimize Doppler signal passage.

15. The method of claim 14, wherein the recession further comprises a fillet surrounding the inner surface.

16. The method of claim 15, wherein the fillet is arranged at a 30 degree angle with respect to the bottom surface.

17. The method of claim 11, wherein:
control signals are sent to the probe on a transmitting line; and
the received reflected ultrasound signal is received on a receiving line.

18. The method of claim 11, wherein the original ultrasound signal is in an ultrasound range near 8 MHz.

19. A flat ultrasound probe, comprising:
means for housing, comprising:
sidewalls, each having a height;
bottom means for contacting an external surface of a patient during operation of the probe, the bottom means comprising:
a width larger than the height of the sidewalls; and
a flat portion; and
recession means in the bottom means on an outer surface of the housing means for containing a means for aiding transmission for aiding in transmission of ultrasound signals, the recession means being rounded on all sides where the recession means contacts the flat portion of the bottom means.

20. The probe of claim 19, further comprising:
ultrasound signal means located inside the housing at an angle for providing an ultrasound signal,
wherein the recession means comprises a geometry based on the angle of the ultrasound signal means.
